(19) United States
(12) Patent Application Publication
(10) Pub. No.: US 2022/0072167 A1
(43) Pub. Date: Mar. 10, 2022

(54) Methods for Treating Cancer Using Combinations of PARP Inhibitors and Antibody Radioconjugates

(71) Applicant: Actinium Pharmaceuticals, Inc., New York, NY (US)

(72) Inventors: Dale Ludwig, Rockaway, NJ (US); Sandesh Seth, New York, NY (US)

(73) Assignee: Actinium Pharmaceuticals, Inc., New York, NY (US)

(21) Appl. No.: 17/419,381

(22) PCT Filed: Jan. 2, 2020
(86) PCT No.: PCT/US2020/012016

§ 371 Date: Jun. 29, 2021

Related U.S. Application Data
(60) Provisional application No. 62/788,206, filed on Jan. 4, 2019.

(51) Int. Cl. A61K 51/10; A61K 31/502; A61K 31/454; A61K 31/55; A61K 31/5025; A61P 35/02

(52) U.S. Cl. CPC A61K 51/1093; A61K 31/502; A61P 35/02; A61K 31/55; A61K 31/5025; A61K 31/454

(57) ABSTRACT

This invention provides a method for treating a subject afflicted with cancer, comprising administering to the subject (i) a PARP inhibitor in conjunction with (ii) a radioisotope-labeled agent that targets cancer cells in the subject, wherein the amounts of the PARP inhibitor and labeled agent, when administered in conjunction with one another, are therapeutically effective. This invention also provides a method for inducing the death of a cancer cell, comprising contacting the cell with (i) a PARP inhibitor in conjunction with (ii) a radioisotope-labeled agent that targets the cancer cell, wherein the amounts of PARP inhibitor and labeled agent, when concurrently contacted with the cell, are effective to induce the cell's death.

This application claims the benefit of U.S. Provisional Application No. 62/788,206, filed Jan. 4, 2019, the contents of which are incorporated herein by reference.

Throughout this application, various publications are cited. The disclosure of these publications is hereby incorporated by reference into this application to describe more fully the state of the art to which this invention pertains.

### FIELD OF THE INVENTION

The present invention relates to treating a subject afflicted with cancer using a therapeutically effective regimen of a PARP inhibitor in conjunction with a radioisotope-labeled agent that targets cancer cells in the subject.

### BACKGROUND OF THE INVENTION

PARP, PARP Inhibitors and Radiation Therapy

Inhibitors of the DNA repair protein “PARP” (poly(ADP-ribose) polymerase), referred to individually and collectively as “PARPi”, have been approved for use in breast and ovarian cancer, particularly in patients having BRCA1/2 mutations. BRCA1 and 2 function in homologous recombination repair (HRR). When mutated, they induce genomic instability by shifting the DNA repair process from conservative and precise HRR to non-fidelitous methods such as DNA end-joining which can produce mutations via deletions and insertions.

PARPi have been shown to exhibit synthetic lethality, as exhibited by potent single agent activity, in BRCA1/2 mutant cells. This essentially blocks repair of single-strand DNA breaks. Since HRR is not functional in these tumor cells, cell death results. Because most tumors do not carry BRCA1 or BRCA2 mutations, the potency of PARPi in such tumors is far less pronounced.

The PARP family of enzymes utilizes beta nicotinamide adenine dinucleotide (13-NAD+) to covalently add Poly(ADP-ribose) (PAR) chains onto target proteins, a process termed “PARylation.” PARP1 (which is the best-studied member) and PARP2, are important components of the DNA damage repair (DDR) pathway. PARP1 is involved in the repair of single-stranded DNA breaks (1), and possibly other DNA lesions (2). Through its zinc finger domains, PARP1 binds to damaged DNA and then PARylates a series of DNA repair effector proteins, releasing nicotinamide as a by-product (2). Subsequently, PARP1 auto-PARylation leads to release of the protein from the DNA.

To date, the FDA has approved four PARP inhibitor drugs (olaparib, niraparib, rucaparib and talazoparib) as monotherapy agents, specifically in patients with germline and somatic mutations in the BRCA1 and BRCA2 genes. Along with veliparib, olaparib, niraparib and rucaparib are among the first generation of PARP inhibitors that entered clinical trials. Their IC₅₀ values are in the nanomolar range. In contrast, second generation PARP inhibitors like talazoparib have IC₅₀ values in the picomolar range.

These PARP inhibitors all bind to the binding site of the cofactor, b nicotinamide adenine dinucleotide (b-NAD+), in the catalytic domain of PARP1 and PARP2. However, they differ in their capability to trap PARP1 on DNA, while such capability seems to correlate with cytotoxicity and drug efficacy. Specifically, drugs like talazoparib and olaparib are more effective in trapping PARP1 than are veliparib (3, 4).

The efficacy of PARP inhibitors in ovarian cancer and breast cancer patients who have loss-of-function mutations in BRCA1 or BRCA2 genes is largely attributed to the genetic concept of synthetic lethality: that proteins of BRCA 1 and 2 normally maintain the integrity of the genome by mediating a DNA repair process, known as homologous recombination (HR); and PARPi causes a persistent DNA lesion that, normally, would otherwise be repaired by HR. In the presence of PARPi, PARP1 is trapped on DNA which stalls progression of the replication fork. This stalling is cytotoxic unless timely repaired by the HR system. In cells lacking effective HR, they are unable to effectively repair these DNA lesions, and thus die (5) (FIG. 7).

Again, mutations in BRCA genes and others in the HR system are not prevalent in many cancer types. So, to better harness the therapeutic benefits of PARP inhibitors in such cancers, one can induce “artificial” synthetic lethality by pairing a PARP inhibitor with either chemotherapy or radiation therapy. Indeed, the original proposed use of PARP inhibitors was as chemo- or radiosensitizing agents (6).

Drean, et al. proposed three broad mechanisms for these combinatorial PARP inhibitor therapies: (1) increased accumulation of DNA damage and subsequent dependence on PARP-mediated DNA damage repair; (2) increased levels of trapped PARP-DNA complexes; and (3) induction of BRCAness phenotype to elicit PARPi/BRCAness synthetic lethality (6) (FIG. 8).

Various PARPi combination-therapy clinical trials are ongoing. Many involve combination with chemotherapies. Radiation therapy (RT), which uses ionizing radiation like alpha and beta particles, X-rays and gamma rays, may have an advantage over chemotherapy in terms of toxicity, given chemotherapy's generally poor reputation in this area.

Further, preclinical studies have demonstrated that combining RT and PARPi can increase the sensitivity of BRCA1/2 mutant tumor cells to PARP inhibition and extend the sensitivity of non-mutant BRCA tumors to PARP inhibition. Additional studies have shown that ionizing radiation (IR) itself can mediate PARPi synthetic lethality in tumor cells. For example, Sizemore and colleagues determined that IR effects cytoplasmic translocation of BRCA1 protein in IR-treated tumor cells, leading to suppression of HRR DNA repair and the induction of synthetic PARPi lethality in wild-type BRCA1 and HR-proficient tumor cells (FIG. 9) (7). The tumor suppressor p53 was identified as a key factor that regulates DNA damage-induced BRCA1 cytoplasmic sequestration following IR (6). A number of clinical trials are testing PARPi and RT combinations for their therapeutic effect.

Antibody Radioconjugates

The term “antibody radioconjugate” (ARC) refers to a source of ionizing radiation (e.g., alpha and beta particles, and gamma rays) linked to a targeting agent (i.e., an antibody). Actinium-225 (Ac-225) is an ideal source of radiation for such purpose. Ac-225 (Linear Energy Transfer=6.83 MeV; T½=10 days; path length 40-80 uM) causes clustered DNA lesions including single-strand breaks (SSBs) and double-strand breaks (DSBs) (8). Even one alpha emission may be lethal to a tumor cell. Importantly, the ARC, once bound to a tumor cell, does not need to enter the cell to kill it. So, the process for effecting cell killing is far simpler than that required for an antibody-drug (chemo) conjugate. In addition, because Ac-225 can emit four alpha particles as it decays over its 10-day half-life, a bound ARC can kill not only the target cell but also adjacent unbound tumor cells, including those that may be target antigen negative. Importantly, the short path length of an alpha emitter limits the field of damage to immediately adjacent cells (i.e., as few as 2-6 cell diameters). As a result, normal tissue is spared.

### SUMMARY OF THE INVENTION

This invention provides a method for treating a subject afflicted with cancer, comprising administering to the subject (i) a PARP inhibitor in conjunction with (ii) a radioisotope-labeled agent that targets cancer cells in the subject, wherein the amounts of the PARP inhibitor and labeled agent, when administered in conjunction with one another, are therapeutically effective.

This invention also provides a method for treating a human subject afflicted with breast cancer, wherein the subject does not possess a deleterious BRCA1/2 mutation, comprising administering to the subject (i) a PARP inhibitor selected from the group consisting of olaparib, niraparib, rucaparib and talazoparib in conjunction with (ii) ²²⁵Ac-labeled trastuzumab, wherein the amounts of the PARP inhibitor and ²²⁵Ac-labeled trastuzumab, when administered in conjunction with one another, are therapeutically effective.

This invention further provides a method for treating a human subject afflicted with acute myeloid leukemia, comprising administering to the subject (i) a PARP inhibitor selected from the group consisting of olaparib, niraparib, rucaparib and talazoparib in conjunction with (ii) ²²⁵Ac-labeled HuM195, wherein the amounts of the PARP inhibitor and ²²⁵Ac-labeled HuM195, when administered in conjunction with one another, are therapeutically effective.

This invention still further provides a method for inducing the death of a cancer cell, comprising contacting the cell with (i) a PARP inhibitor in conjunction with (ii) a radioisotope-labeled agent that targets the cancer cell, wherein the amounts of PARP inhibitor and radiolabeled agent, when contacted with the cell in conjunction with one another, are effective to induce the cell's death.

This invention still further provides a method for inducing the death of a human breast cancer cell that does not possess a deleterious BRCA1/2 mutation, comprising contacting the cell with (i) a PARP inhibitor selected from the group consisting of olaparib, niraparib, rucaparib and talazoparib in conjunction with (ii) ²²⁵Ac-labeled trastuzumab, wherein the amounts of PARP inhibitor and ²²⁵Ac-labeled trastuzumab, when contacted with the cell in conjunction with one another, are effective to induce the cell's death.

This invention also provides a method for inducing the death of an acute myeloid leukemic cell, comprising contacting the cell with (i) a PARP inhibitor selected from the group consisting of olaparib, niraparib, rucaparib and talazoparib in conjunction with (ii) ²²⁵Ac-labeled HuM195, wherein the amounts of PARP inhibitor and ²²⁵Ac-labeled HuM195, when contacted with the cell in conjunction with one another, are effective to induce the cell's death.

Finally, this invention provides an anti-HER2 antibody labeled with a radioisotope, such as ²²⁵Ac-labeled trastuzumab.

### BRIEF DESCRIPTION OF THE FIGURES

FIG. 1

This figure shows a schematic diagram of the expression plasmids for HuM195.

The humanized VL and VH exons of HuM195 are flanked by XbaI sites. The VL exon was inserted into mammalian expression vector pVk, and the VH exon into pVg1 (Co, et al., J. Immunol. 148:1149-1154, 1992).

FIG. 2

This figure shows the complete sequence of the HuM195 light chain gene cloned in pVk between the XbaI and BamHI sites. The nucleotide number indicates its position in the plasmid pVk-HuM195. The VL and CK exons are translated in single letter code; the dot indicates the translation termination codon. The mature light chain begins at the double-underlined aspartic acid (D). The intron sequence is in italics. The polyA signal is underlined.

FIG. 3

This figure shows the complete sequence of the HuM195 heavy chain gene cloned in pVg1 between the XbaI and BamHI sites. The nucleotide number indicates its position in the plasmid pVg1-HuM195. The VH, CH1, H, CH2 and CH3 exons are translated in single letter code; the dot indicates the translation termination codon. The mature heavy chain begins at the double-underlined glutamine (Q). The intron sequences are in italics. The polyA signal is underlined.

FIG. 4

This figure shows the structure of ²²⁵Ac-Lintuzumab (²²⁵Ac-HuM195).

FIG. 5

This figure shows a flowchart for the production of ²²⁵Ac-HuM195.

FIG. 6

This figure shows a dosing protocol for ²²⁵Ac-Lintuzumab (²²⁵Ac-HuM195) treatment of AML, without PARPi.

FIG. 7

This figure shows a schematic of PARPi function in relation to HRR.

FIG. 8

This figure shows three broad mechanisms for the combinatorial PARP inhibitor therapies, as proposed by Drean, et al. (6) (Figure taken from reference.).

FIG. 9

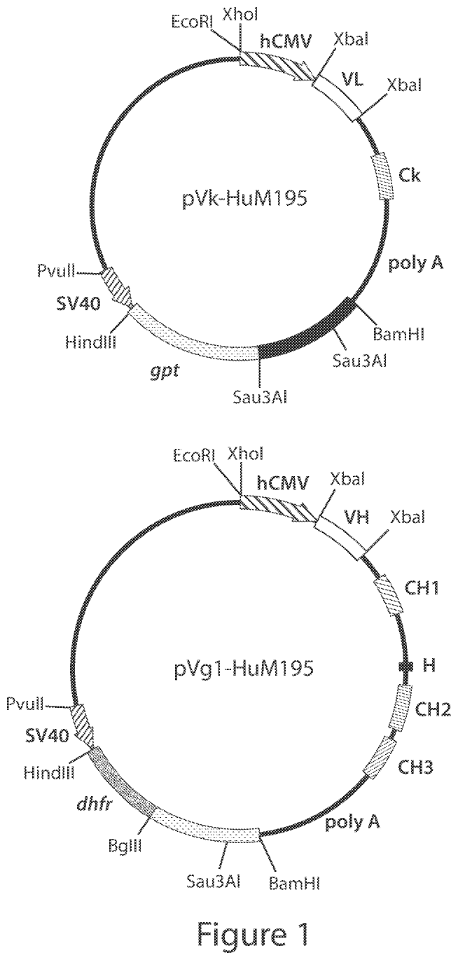

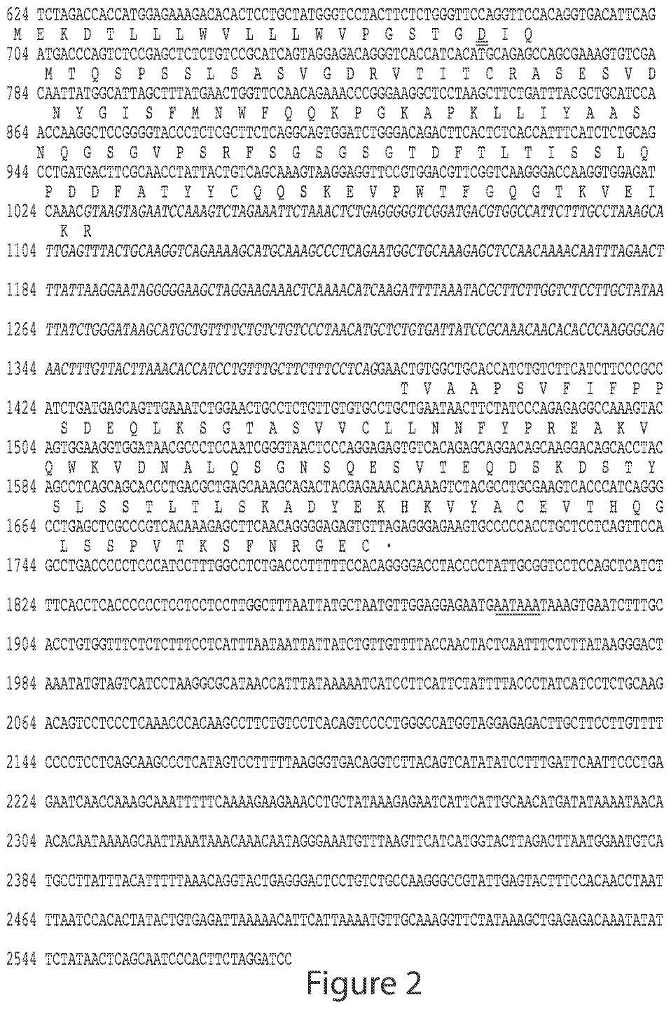

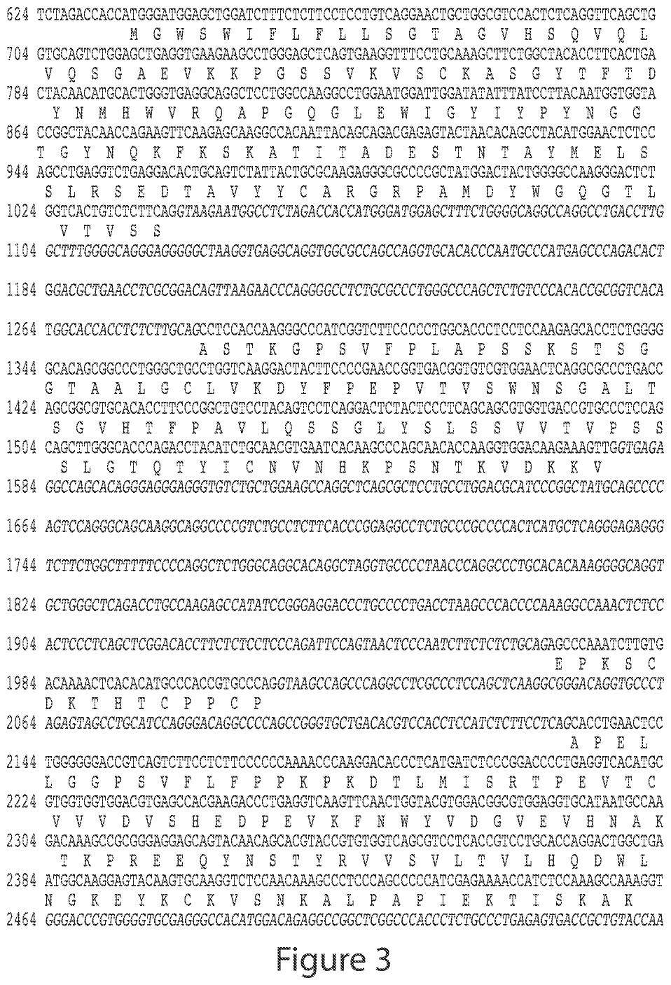

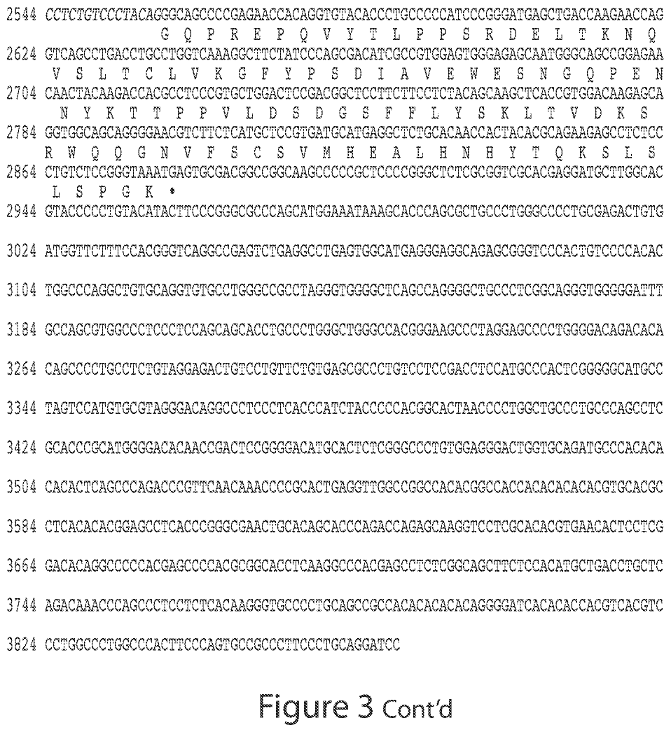

This figure shows that IR sensitizes glioblastoma cancer cells to PARPi. The lines represent treatment with either nothing (control), ionizing radiation (IR), veliparib (ABT-888), or veliparib plus ionizing radiation (ABT-888+IR). (7)

### DETAILED DESCRIPTION OF THE INVENTION

This invention provides methods for treating a subject afflicted with cancer. These methods comprise administering to the subject two types of agents in conjunction with one another. The first type of agent is a PARP inhibitor such as olaparib, niraparib, rucaparib or talazoparib. The second type is a radioisotope-labeled agent, such as ²²⁵Ac-labeled trastuzumab or ²²⁵Ac-labeled HuM195, that targets cancer cells in the subject.

### Definitions

In this application, certain terms are used which shall have the meanings set forth as follows.

As used herein, “administer”, with respect to an agent, means to deliver the agent to a subject's body via any known method. Specific modes of administration include, without limitation, intravenous, oral, sublingual, transdermal, subcutaneous, intraperitoneal, intrathecal and intra-tumoral administration. Preferably, PARP inhibitors are administered orally, and antibody radioconjugates are administered intravenously.

In addition, in this invention, the various PARP inhibitors, antibodies and other antigen-targeting agents used can be formulated using one or more routinely used pharmaceutically acceptable carriers. Such carriers are well known to those skilled in the art. For example, injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Likewise, oral delivery systems include, for example, tablets and capsules. These can contain excipients such as binders (e.g., hydroxypropylmethyl-cellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

As used herein, the term “agent”, whether in reference to a PARP inhibitor or a radioisotope-labeled agent, can be any type of compound or composition useful for such purpose. Types of agents include, without limitation, antibodies, other protein-based drugs, peptides, nucleic acids, carbohydrates and small molecules drugs.

As used herein, the term “antibody” includes, without limitation, (a) an immunoglobulin molecule comprising two heavy chains and two light chains and which recognizes an antigen; (b) polyclonal and monoclonal immunoglobulin molecules; (c) monovalent and divalent fragments thereof, and (d) bi-specific forms thereof. Immunoglobulin molecules may derive from any of the commonly known classes, including but not limited to IgA, secretory IgA, IgG and IgM. IgG subclasses are also well known to those in the art and include, but are not limited to, human IgG1, IgG2, IgG3 and IgG4. Antibodies can be both naturally occurring and non-naturally occurring. Furthermore, antibodies include chimeric antibodies, wholly synthetic antibodies, single chain antibodies, and fragments thereof. Antibodies may be human, humanized or nonhuman.

As used herein, an “anti-CD33 antibody” is an antibody that binds to any available epitope of CD33. In one embodiment, the anti-CD33 antibody binds to the epitope recognized by the antibody HuM195.

A “hematologic malignancy”, also known as a blood cancer, is a cancer that originates in blood-forming tissue, such as the bone marrow or other cells of the immune system. Hematologic malignancies include, without limitation, leukemias (such as acute myeloid leukemia (AML), acute promyelocytic leukemia, acute lymphoblastic leukemia, acute mixed lineage leukemia, chronic myeloid leukemia, chronic lymphocytic leukemia, hairy cell leukemia and large granular lymphocytic leukemia), myelodysplastic syndrome (MDS), myeloproliferative disorders (polycythemia vera, essential thrombocytosis, primary myelofibrosis and chronic myeloid leukemia), lymphomas (e.g., Hodgkin's lymphoma and non-Hodgkin's lymphoma), multiple myeloma, and MGUS and similar disorders. Hematologic malignancies are characterized by hematologic malignancy-associated antigens. Such antigen can be, for example, a protein and/or carbohydrate marker found exclusively or predominantly on the surface of a cancer cell associated with that particular malignancy. Examples of hematologic malignancy-associated antigens include, without limitation, CD20, CD33, CD38, CD45, CD52, CD123 and CD319.

The antibody “HuM195” (also known as lintuzumab) is known, as are methods of making it. Likewise, methods of labeling HuM195 with ²²⁵Ac are known. These methods are exemplified, for example, in Scheinberg, et al., U.S. Pat. No. 6,683,162. This information is also exemplified in the examples and figures below.

As used herein, administering to a subject a PARP inhibitor “in conjunction with” a radioisotope-labeled agent that targets cancer cells in the subject means administering the PARP inhibitor before, during and/or after administration of the labeled agent. This administration includes, without limitation, the following scenarios: (i) the PARP inhibitor is administered first, and the labeled agent is administered second; (ii) the PARP inhibitor is administered concurrently with the labeled agent (e.g., the PARP inhibitor is administered orally once per day for n days, and the labeled agent is administered intravenously in a single dose on one of days 2 through n−1 of the PARP inhibitor regimen); (iii) the PARP inhibitor is administered concurrently with the labeled agent (e.g., the PARP inhibitor is administered orally for a duration of greater than one month (e.g., orally once per day for 35 days, 42 days, 49 days, or a longer period during which the cancer being treated does not progress and during which the PARP inhibitor does not cause unacceptable toxicity), and the labeled agent is administered intravenously in a single dose on a day within the first month of the PARP inhibitor regimen); and (iv) the labeled agent is administered first (e.g., intravenously in a single dose or a plurality of doses over a period of weeks), and the PARP inhibitor is administered second (e.g., orally once per day for 21 days, 28 days, 35 days, 42 days, 49 days, or a longer period during which the cancer being treated does not progress and during which the PARP inhibitor does not cause unacceptable toxicity). Additional permutations are provided below in the Examples section.

As used herein, “inducing” the death of a cancer cell includes, without limitation, (i) directly causing the cell's death, and (ii) indirectly causing the cell's death (e.g., by triggering a cascade of biochemical events that ultimately leads to the cell's death).

As used herein, a “radioisotope” can be an alpha-emitting isotope, a beta-emitting isotope, and/or a gamma-emitting isotope. Examples of radioisotopes include the following: ⁹⁰Y, ⁸⁹Sr, ¹⁵³Sm, ³²P, ²²⁵Ac, ²¹³Bi, ²¹³Po, ²¹¹At, ²¹²Bi, ²¹³Bi, ²²³Ra ²²⁷Th, ¹⁴⁹Tb, ¹³¹I, ¹³⁷Cs, ²¹²Pb and ¹⁰³Pd. Thus, the radiolabeled antibodies envisioned in this invention include, without limitation, ⁹⁰Y-HuM195, ⁸⁹Sr-HuM195, ¹⁵³Sm-HuM195, ³²P-HuM195, ²²⁵Ac-HuM195, ²¹³Bi-HuM195, ²¹³Po-HuM195, ²¹¹At-HuM195, ²¹²Bi-HuM195, ²¹³Bi-HuM195, ²²³Ra-HuM195, ²²⁷Th-HuM195, ¹⁴⁹Tb-HuM195, ¹³¹I-HuM195, ¹³⁷Cs-HuM195, ²¹²Pb-HuM195 and ¹⁰³Pd-HuM195, ⁹⁰Y-trastuzumab, ⁸⁹Sr-trastuzumab, ¹⁵³Sm-trastuzumab, ³²P-trastuzumab, ²²⁵Ac-trastuzumab, ²¹³Bi-trastuzumab, ²¹³Po-trastuzumab, ²¹¹At-trastuzumab, ²¹²Bi-trastuzumab, ²¹³Bi-trastuzumab, ²²³Ra-trastuzumab, ²²⁷Th-trastuzumab, ¹⁴⁹Tb-trastuzumab, ¹³¹I-trastuzumab, ¹³⁷Cs-trastuzumab, ²¹²Pb-trastuzumab and ¹⁰³Pd-trastuzumab. Each of the antibody radioconjugates above is also envisioned, mutatis mutandis, and without limitation, for each of the following antibodies: alemtuzumab (Campath®), ibritumomab tiuxetan (Zevalin®), brentuximab vedotin (Adcetris®) and trastuzumab emtansine (Kadcyla®). Methods for affixing a radioisotope to an antibody (i.e., “labeling” an antibody with a radioisotope) are known.

As used herein, the term “subject” includes, without limitation, a mammal such as a human, a non-human primate, a dog, a cat, a horse, a sheep, a goat, a cow, a rabbit, a pig, a rat and a mouse. Where the subject is human, the subject can be of any age. For example, the subject can be 60 years or older, 65 or older, 70 or older, 75 or older, 80 or older, 85 or older, or 90 or older. Alternatively, the subject can be 50 years or younger, 45 or younger, 40 or younger, 35 or younger, 30 or younger, 25 or younger, or 20 or younger. For a human subject afflicted with AML, MDS or MM, the subject can be newly diagnosed, or relapsed and/or refractory, or in remission.

As used herein, an amount of PARP inhibitor and an amount of radioisotope-labeled agent that targets cancer cells in the subject, when administered in conjunction with each other, are “therapeutically effective” if the subject is treated.

The antibody “trastuzumab” (also known as Herceptin®) is known, as are methods of making it.

As used herein, “treating” a subject afflicted with a disorder shall include, without limitation, (i) slowing, stopping or reversing the disorder's progression, (ii) slowing, stopping or reversing the progression of the disorder's symptoms, (iii) reducing the likelihood of the disorder's recurrence, and/or (iv) reducing the likelihood that the disorder's symptoms will recur. In the preferred embodiment, treating a subject afflicted with a disorder means (i) reversing the disorder's progression, ideally to the point of eliminating the disorder, and/or (ii) reversing the progression of the disorder's symptoms, ideally to the point of eliminating the symptoms, and/or (iii) reducing or eliminating the likelihood of relapse (i.e., consolidation, which is a common goal of post-remission therapy for AML and, ideally, results in the destruction of any remaining leukemia cells).

The treatment of hematologic malignancy, such as the treatment of AML, can be measured according to a number of clinical endpoints. These include, without limitation, survival time (such as weeks, months or years of improved survival time, e.g., one, two or more months' of additional survival time), and response status (such as complete remission (CR), complete remission with incomplete platelet recovery (CRp), complete remission with incomplete peripheral blood recovery (CRi), morphologic leukemia-free state (MLFS) and partial remission (PR)).

In one embodiment, treatment of hematologic malignancy, such as the treatment of AML, can be measured in terms of remission. Included here are the following non-limiting examples. (1) Morphologic complete remission (“CR”): ANC≥1,000/mcl, platelet count≥100,000/mcl, <5% bone marrow blasts, no Auer rods, no evidence of extramedullary disease. (No requirements for marrow cellularity, hemoglobin concentration). (2) Morphologic complete remission with incomplete blood count recovery (“CRi”): Same as CR but ANC may be <1,000/mcl and/or platelet count<100,000/mcl. (3) Partial remission (PR): ANC≥1,000/mcl, platelet count>100,000/mcl, and at least a 50% decrease in the percentage of marrow aspirate blasts to 5-25%, or marrow blasts<5% with persistent Auer rods. These criteria and others are known, and are described, for example, in SWOG Oncology Research Professional (ORP) Manual Volume I, Chapter 11A, Leukemia (2014).

### EMBODIMENTS OF THE INVENTION

This invention combines the use of two different agents to treat cancer. Here, PARP inhibitors are administered in conjunction with antibody radioconjugates to more effectively treat patients with solid tumors as well as hematologic malignancies.

Specifically, this invention provides a method for treating a subject afflicted with cancer, comprising administering to the subject (i) a PARP inhibitor in conjunction with (ii) a radioisotope-labeled agent that targets cancer cells in the subject, wherein the amounts of the PARP inhibitor and labeled agent, when administered in conjunction with one another, are therapeutically effective. Preferably, the subject is human.

In one embodiment of this therapeutic method, the cancer is a solid tumor. Solid tumors include, without limitation, breast cancer, ovarian cancer, prostate cancer, lung cancer, squamous cell carcinoma of the head and neck, gastric cancer, pancreatic cancer, brain cancer (e.g., glioblastoma and neuroblastoma), liver cancer, sarcoma and melanoma. Preferably, the solid tumor is breast cancer or ovarian cancer. In one embodiment, the subject possesses a deleterious BRCA1/2 mutation. Preferably, though, the subject does not possess a deleterious BRCA1/2 mutation.

In the various embodiments of this invention, the PARP inhibitor may be any known agent performing that function, and preferably, one approved by the FDA. Preferably, the PARP inhibitor is olaparib (Lynparza®), niraparib (Zejula®), rucaparib (Rubraca®) or talazoparib (Talzenna®).

In the various embodiments of this invention, the radioisotope-labeled agent may be any known agent that targets cancer cells, and preferably, one approved by the FDA. In another preferred embodiment of this method, the radioisotope-labeled agent is an anti-HER2 antibody labeled with an alpha-emitting isotope. Preferably, the radioisotope-labeled agent is ²²⁵Ac-labeled trastuzumab (also referred to herein as ²²⁵Ac-trastuzumab).

In another embodiment of this therapeutic method, the cancer is a hematologic malignancy. Preferably, the hematologic malignancy is acute myeloid leukemia, myelodysplastic syndrome or multiple myeloma. In another preferred embodiment, the radioisotope-labeled agent is an anti-CD33 antibody labeled with an alpha-emitting isotope. Preferably, the radioisotope-labeled agent is ²²⁵Ac-labeled HuM195 (also referred to herein as ²²⁵Ac-HuM195).

This invention also provides a method for treating a human subject afflicted with breast cancer, wherein the subject does not possess a deleterious BRCA1/2 mutation, comprising administering to the subject (i) a PARP inhibitor selected from the group consisting of olaparib, niraparib, rucaparib and talazoparib in conjunction with (ii) ²²⁵Ac-labeled trastuzumab, wherein the amounts of the PARP inhibitor and ²²⁵Ac-labeled trastuzumab, when administered in conjunction with one another, are therapeutically effective. Specifically envisioned in this method are the following combinations: (i) ²²⁵Ac-labeled trastuzumab and olaparib; (ii) ²²⁵Ac-labeled trastuzumab and niraparib; (iii) ²²⁵Ac-labeled trastuzumab and rucaparib; and (iv) ²²⁵Ac-labeled trastuzumab and talazoparib.

This invention further provides a method for treating a human subject afflicted with acute myeloid leukemia, comprising administering to the subject (i) a PARP inhibitor selected from the group consisting of olaparib, niraparib, rucaparib and talazoparib in conjunction with (ii) ²²⁵Ac-labeled HuM195, wherein the amounts of the PARP inhibitor and ²²⁵Ac-labeled HuM195, when administered in conjunction with one another, are therapeutically effective. Specifically envisioned in this method are the following combinations: (i) ²²⁵Ac-labeled HuM195 and olaparib; (ii) ²²⁵Ac-labeled HuM195 and niraparib; (iii) ²²⁵Ac-labeled HuM195 and rucaparib; and (iv) ²²⁵Ac-labeled HuM195 and talazoparib.

This invention still further provides a method for inducing the death of a cancer cell, comprising contacting the cell with (i) a PARP inhibitor in conjunction with (ii) a radioisotope-labeled agent that targets the cancer cell, wherein the amounts of PARP inhibitor and radiolabeled agent, when contacted with the cell in conjunction with one another, are effective to induce the cell's death. Preferably, the cancer cell is a human cancer cell.

In one embodiment of this method, the cancer cell is a solid tumor cell. Solid tumor cells include, without limitation, breast cancer cells, ovarian cancer cells, prostate cancer cells, lung cancer cells, cells of squamous cell carcinoma of the head and neck, gastric cancer cells, pancreatic cancer cells, brain cancer cells, liver cancer cells, sarcoma cells and melanoma cells. Preferably, the tumor cell is a breast cancer cell or ovarian cancer cell. In one embodiment, this tumor cell possesses a deleterious BRCA1/2 mutation. Preferably, though, it does not. Also, the PARP inhibitor preferably is olaparib, niraparib, rucaparib or talazoparib. In still another preferred embodiment of this method, the radioisotope-labeled agent is an anti-HER2 antibody labeled with an alpha-emitting isotope (preferably, the agent is ²²⁵Ac-labeled trastuzumab).

In another embodiment of this therapeutic method, the cancer cell is a hematologic cancer cell. Preferably, the hematologic cancer cell is an acute myeloid leukemic cell, a myelodysplastic syndrome cell, or a multiple myeloma cell. In a preferred embodiment, the PARP inhibitor is olaparib, niraparib, rucaparib or talazoparib. In another preferred embodiment, the radioisotope-labeled agent is ²²⁵Ac-labeled HuM195.

This invention still further provides a method for inducing the death of a human breast cancer cell that does not possess a deleterious BRCA1/2 mutation, comprising contacting the cell with (i) a PARP inhibitor selected from the group consisting of olaparib, niraparib, rucaparib and talazoparib in conjunction with (ii) ²²⁵Ac-labeled trastuzumab, wherein the amounts of PARP inhibitor and ²²⁵Ac-labeled trastuzumab, when contacted with the cell in conjunction with one another, are effective to induce the cell's death. Specifically envisioned in this method are the following combinations: (i) ²²⁵Ac-labeled trastuzumab and olaparib; (ii) ²²⁵Ac-labeled trastuzumab and niraparib; (iii) ²²⁵Ac-labeled trastuzumab and rucaparib; and (iv) ²²⁵Ac-labeled trastuzumab and talazoparib.

This invention also provides a method for inducing the death of an acute myeloid leukemic cell, comprising contacting the cell with (i) a PARP inhibitor selected from the group consisting of olaparib, niraparib, rucaparib and talazoparib in conjunction with (ii) ²²⁵Ac-labeled HuM195, wherein the amounts of PARP inhibitor and ²²⁵Ac-labeled HuM195, when contacted with the cell in conjunction with one another, are effective to induce the cell's death. Specifically envisioned in this method are the following combinations: (i) ²²⁵Ac-labeled HuM195 and olaparib; (ii) ²²⁵Ac-labeled HuM195 and niraparib; (iii) ²²⁵Ac-labeled HuM195 and rucaparib; and (iv) ²²⁵Ac-labeled HuM195 and talazoparib.

In the instant methods, the PARP inhibitor, the labeled agent, or both, are preferably administered in doses that are less than, and/or in dosing regimens of shorter duration than, those presently prescribed on their respective labels. Embodiments of the invention in this regard are set forth in the examples section.

This invention also provides an anti-HER2 antibody labeled with a radioisotope. Preferably, the antibody is ²²⁵Ac-labeled trastuzumab. This invention also provides a pharmaceutical composition comprising this antibody (preferably ²²⁵Ac-labeled trastuzumab) and a pharmaceutically acceptable carrier.

Finally, this invention provides four articles of manufacture. The first article comprises (i) a PARP inhibitor (e.g., olaparib, niraparib, rucaparib or talazoparib) and (ii) a label instructing the user (e.g., a healthcare provider) to treat a subject (e.g., a human) afflicted with a solid tumor (e.g., breast or ovarian cancer in a subject not possessing a deleterious BRCA1/2 mutation) by administering the PARP inhibitor to the subject in conjunction with a radioisotope-labeled agent (e.g., ²²⁵Ac-labeled trastuzumab) that targets cancer cells in the subject, wherein the amounts of the PARP inhibitor and labeled agent, when administered in conjunction with one another, are therapeutically effective. Specifically envisioned in this article are the following combinations: (i) ²²⁵Ac-labeled trastuzumab and olaparib; (ii) ²²⁵Ac-labeled trastuzumab and niraparib; (iii) ²²⁵Ac-labeled trastuzumab and rucaparib; and/or (iv) ²²⁵Ac-labeled trastuzumab and talazoparib.

The second article comprises (i) a PARP inhibitor (e.g., olaparib, niraparib, rucaparib or talazoparib) and (ii) a label instructing the user (e.g., a healthcare provider) to treat a subject (e.g., a human) afflicted with a hematologic malignancy (e.g., acute myeloid leukemia, myelodysplastic syndrome or multiple myeloma) by administering the PARP inhibitor to the subject in conjunction with a radioisotope-labeled agent (e.g., ²²⁵Ac-labeled HuM195) that targets cancer cells in the subject, wherein the amounts of the PARP inhibitor and labeled agent, when administered in conjunction with one another, are therapeutically effective. Specifically envisioned in this article are the following combinations: (i) ²²⁵Ac-labeled HuM195 and olaparib; (ii) ²²⁵Ac-labeled HuM195 and niraparib; (iii) ²²⁵Ac-labeled HuM195 and rucaparib; and/or (iv) ²²⁵Ac-labeled HuM195 and talazoparib.

The third article comprises (i) a radioisotope-labeled agent (e.g., ²²⁵Ac-labeled trastuzumab) that targets cancer cells and (ii) a label instructing the user (e.g., a healthcare provider) to treat a subject (e.g., a human) afflicted with a solid tumor (e.g., breast or ovarian cancer in a subject not possessing a deleterious BRCA1/2 mutation) by administering the labeled agent to the subject in conjunction with a PARP inhibitor (e.g., olaparib, niraparib, rucaparib or talazoparib), wherein the amounts of the PARP inhibitor and labeled agent, when administered in conjunction with one another, are therapeutically effective. Specifically envisioned in this article are the following combinations: (i) ²²⁵Ac-labeled trastuzumab and olaparib; (ii) ²²⁵Ac-labeled trastuzumab and niraparib; (iii) ²²⁵Ac-labeled trastuzumab and rucaparib; and/or (iv) ²²⁵Ac-labeled trastuzumab and talazoparib.

The fourth article comprises (i) a radioisotope-labeled agent (e.g., ²²⁵Ac-labeled HuM195) that targets cancer cells and (ii) a label instructing the user (e.g., a healthcare provider) to treat a subject (e.g., a human) afflicted with a hematologic malignancy (e.g., acute myeloid leukemia, myelodysplastic syndrome or multiple myeloma) by administering the labeled agent to the subject in conjunction with a PARP inhibitor (e.g., olaparib, niraparib, rucaparib or talazoparib), wherein the amounts of the PARP inhibitor and labeled agent, when administered in conjunction with one another, are therapeutically effective. Specifically envisioned in this article are the following combinations: (i) ²²⁵Ac-labeled HuM195 and olaparib; (ii) ²²⁵Ac-labeled HuM195 and niraparib; (iii) ²²⁵Ac-labeled HuM195 and rucaparib; and/or (iv) ²²⁵Ac-labeled HuM195 and talazoparib.

This invention will be better understood by reference to the examples which follow, but those skilled in the art will readily appreciate that the specific examples detailed are only illustrative of the invention as described more fully in the claims which follow thereafter.

### EXAMPLES

### Example 1—Structure of ²²⁵Ac-Lintuzumab (²²⁵Ac-HuM195)

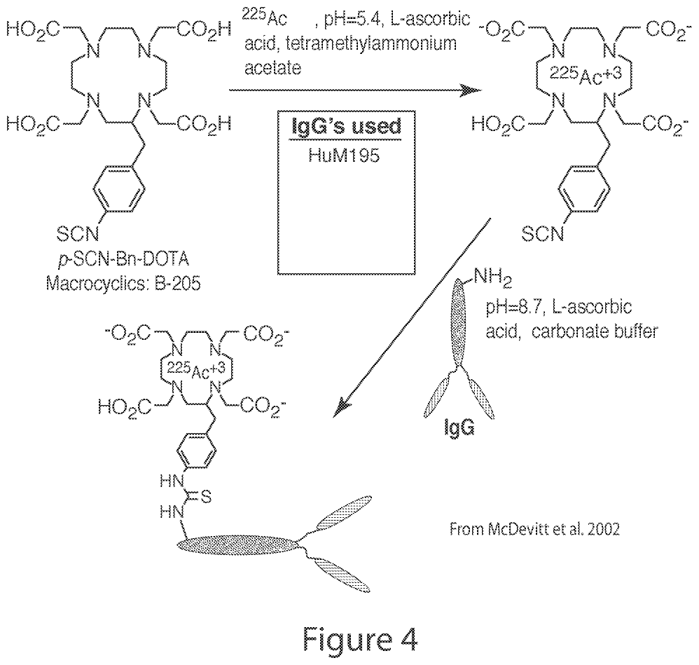

²²⁵Ac-Lintuzumab includes three key components; humanized monoclonal antibody HuM195 (generic name, lintuzumab), the alpha-emitting radioisotope ²²⁵Ac, and the bi-functional chelate 2-(p-isothiocyanatobenzyl)-1,4,7,10-tetraazacyclododecane-1,4,7,10-tetraacetic acid (p-SCN-Bn-DOTA). As depicted in FIG. 4, HuM195 is radiolabeled using the bi-functional chelate p-SCN-Bn-DOTA that binds to ²²⁵Ac and that is covalently attached to the IgG via a lysine residue on the antibody.

### Example 2—p-SCN-Bn-DOTA

DOTA, 2-(4-Isothiocyanatobenzyl)-1,4,7,10-tetraazacyclododecane tetraacetic acid (Macrocyclics item code B205-GMP) is synthesized by a multi-step organic synthesis that is fully described in U.S. Pat. No. 4,923,985.

### Example 3—Preparation of ²²⁵Ac-Lintuzumab (²²⁵Ac-HuM195)

The procedure for preparing ²²⁵Ac-Lintuzumab is based on the method described by Michael R. McDevitt, “Design and synthesis of ²²⁵Ac radioimmuno-pharmaceuticals, Applied Radiation and Isotope”, 57 (2002), 841-847. The procedure involves radiolabeling the bi-functional chelate, p-SCN-Bn-DOTA, with the radioisotope ²²⁵Ac, followed by binding of the radiolabeled p-SCN-Bn-DOTA to the antibody (HuM195). The construct, ²²⁵Ac-p-SCN-Bn-DOTA-HuM195, is purified using 10 DG size exclusion chromatography and eluted with 1% human serum albumin (HSA). The resulting drug product, Ac225-Lintuzumab, is then passed through a 0.2 μm sterilizing filter.

### Example 4—Process Flow for Preparation of ²²⁵Ac-Lintuzumab (²²⁵Ac-HuM195)

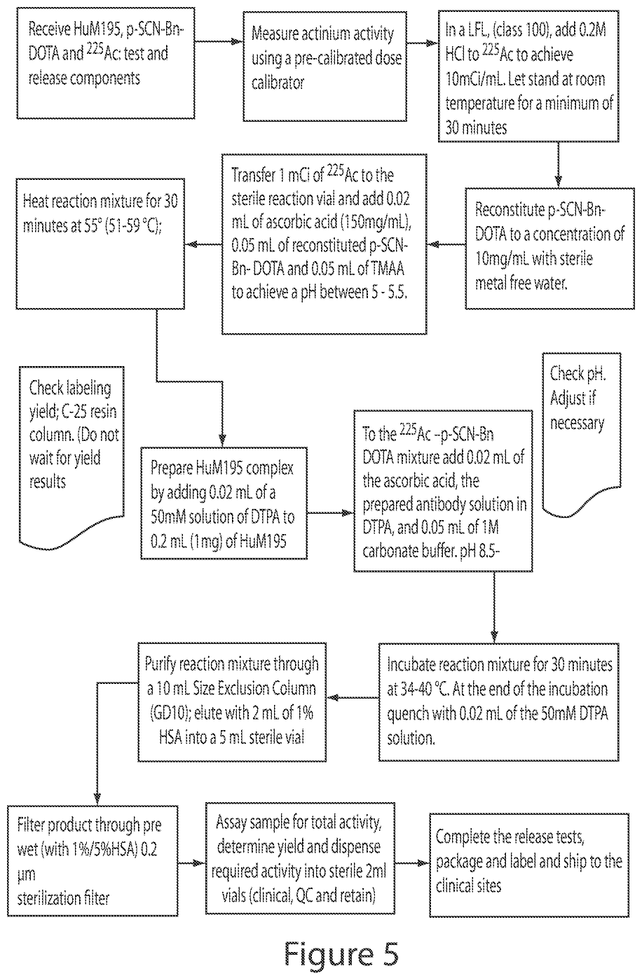

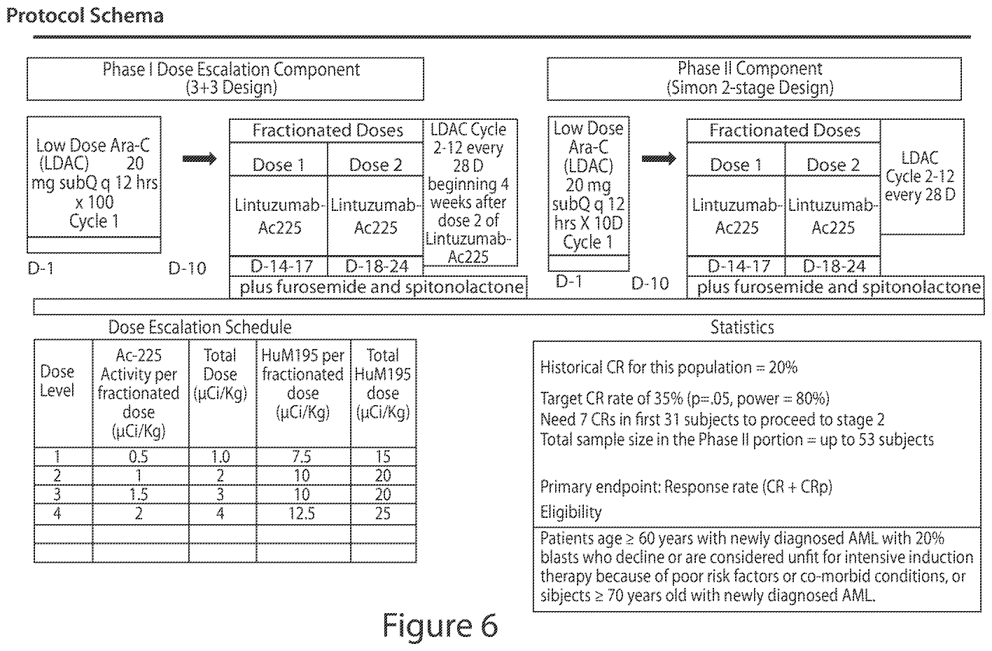

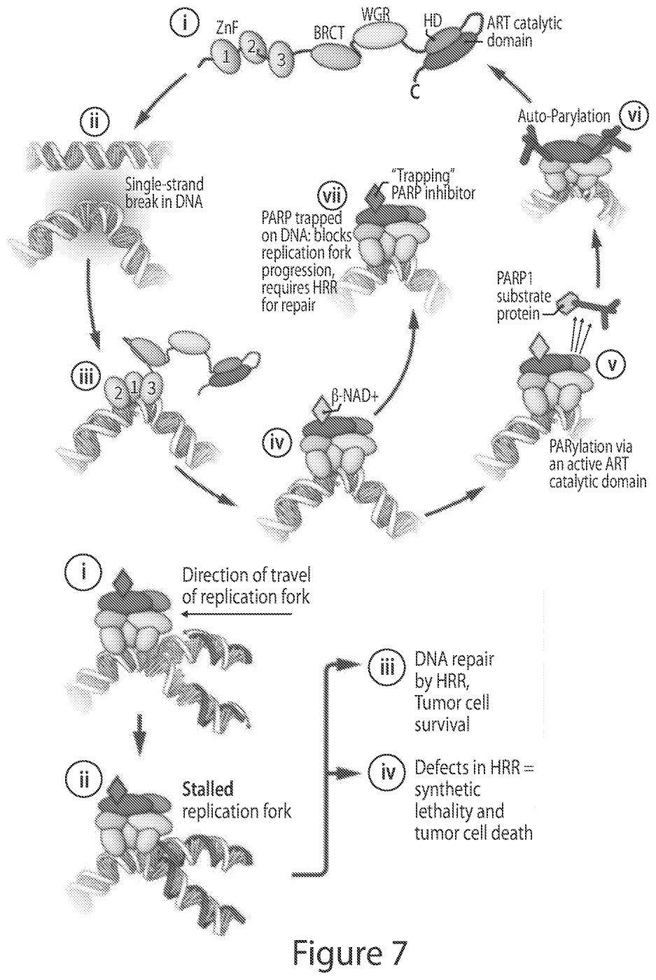

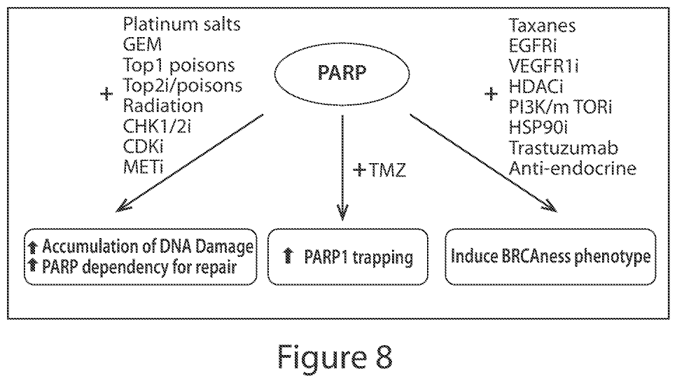

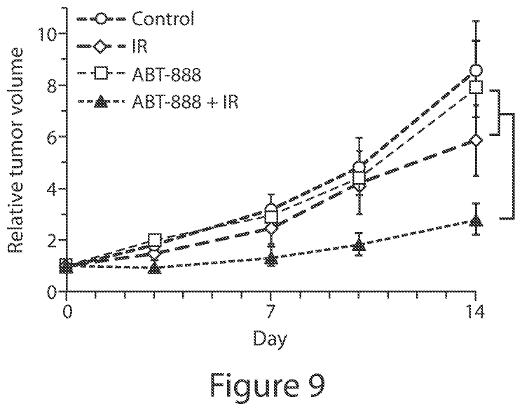

The procedure, shown in FIG. 5, begins with confirming the identity of all components and the subsequent QC release of the components to production. The ²²⁵Ac is assayed to confirm the level of activity and is reconstituted to the desired activity concentration with hydrochloric acid. A vial of lyophilized p-SCN-Bn-DOTA is reconstituted with metal-free water to a concentration of 10 mg/mL. To the actinium reaction vial, 0.02 ml of ascorbic acid solution (150 mg/mL) and 0.05 ml of reconstituted p-SCN-Bn-DOTA are added and the pH adjusted to between 5 and 5.5 with 2M tetramethylammonium acetate (TMAA). The mixture is then heated at 55±4° C. for 30 minutes.

To determine the labeling efficiency of the ²²⁵Ac-p-SCN-Bn-DOTA, an aliquot of the reaction mixture is removed and applied to a 1 ml column of Sephadex C25 cation exchange resin. The product is eluted in 2-4 ml fractions with a 0.9% saline solution. The fraction of ²²⁵Ac activity that elutes is ²²⁵Ac-p-SCN-Bn-DOTA and the fraction that is retained on the column is un-chelated, unreactive ²²⁵Ac. Typically, the labeling efficiency is greater than 95%.

To the reaction mixture, 0.22 ml of previously prepared HuM195 in DTPA (1 mg HuM195) and 0.02 ml of ascorbic acid are added. The DTPA is added to bind any trace amounts of metals that may compete with the labeling of the antibody.

The ascorbic acid is added as a radio-protectant. The pH is adjusted with carbonate buffer to pH 8.5-9. The mixture is heated at 37±3° C. for 30 minutes.

The final product is purified by size exclusion chromatography using 10DG resin and eluted with 2 ml of 1% HSA. Typical reaction yields are 10%.

### Example 5—Olaparib (Lynparza®)—Normal and Reduced Dosing Regimens

Olaparib is sold by AstraZeneca under the brand name Lynparza®. Lynparza® is sold in tablet form at 100 mg and 150 mg. The dosage is 300 mg taken orally twice daily for a daily total of 600 mg. Dosing continues until disease progression or unacceptable toxicity. This dosing regimen is referred to herein as the “normal” human dosing regimen for Lynparza®, regardless of the disorder treated. Any dosing regimen having a shorter duration (e.g., 21 days) or involving the administration of less Lynparza® (e.g., 300 mg/day) is referred to herein as a “reduced” human dosing regimen. Examples of reduced human dosing regimens include the following: (i) 550 mg/day; (ii) 500 mg/day; (iii) 450 mg/day; (iv) 400 mg/day; (v) 350 mg/day; (vi) 300 mg/day; (vii) 250 mg/day; (viii) 200 mg/day; (ix) 150 mg/day; (x) 100 mg/day; or (xi) 50 mg/day.

### Example 6—Niraparib (Zeiula®)—Normal and Reduced Dosing Regimens

Niraparib is sold by Tesaro under the brand name Zejula®. Zejula® is sold in capsule form at 100 mg. The dosage is 300 mg taken orally once daily. Dosing continues until disease progression or unacceptable adverse reaction. This dosing regimen is referred to herein as the “normal” human dosing regimen for Zejula®, regardless of the disorder treated. Any dosing regimen having a shorter duration (e.g., 21 days) or involving the administration of less Zejula® (e.g., 150 mg/day) is referred to herein as a “reduced” human dosing regimen. Examples of reduced human dosing regimens include the following: (i) 250 mg/day; (ii) 200 mg/day; (iii) 150 mg/day; (iv) 100 mg/day; or (v) 50 mg/day.

### Example 7—Rucaparib (Rubraca®)—Normal and Reduced Dosing Regimens

Rucaparib is sold by Clovis Oncology, Inc. under the brand name Rubraca™ Rubraca™ is sold in tablet form at 200 mg and 300 mg. The dosage is 600 mg taken orally twice daily for a daily total of 1,200 mg. Dosing continues until disease progression or unacceptable toxicity. This dosing regimen is referred to herein as the “normal” human dosing regimen for Rubraca™, regardless of the disorder treated. Any dosing regimen having a shorter duration (e.g., 21 days) or involving the administration of less Rubraca™ (e.g., 600 mg/day) is referred to herein as a “reduced” human dosing regimen. Examples of reduced human dosing regimens include the following: (i) 1,150 mg/day; (ii) 1,100 mg/day; (iii) 1,050 mg/day; (iv) 1,000 mg/day; (v) 950 mg/day; (vi) 900 mg/day; (vii) 850 mg/day; (viii) 800 mg/day; (ix) 750 mg/day; (x) 700 mg/day; (xi) 650 mg/day; (xii) 600 mg/day; (xiii) 550 mg/day; (xiv) 500 mg/day; (xv) 450 mg/day; (xvi) 400 mg/day; (xvii) 350 mg/day; (xviii) 300 mg/day; (xix) 250 mg/day; (xx) 200 mg/day; (xxi) 150 mg/day; or (xxii) 100 mg/day.

### Example 8—Talazoparib (Talzenna™)—Normal and Reduced Dosing Regimens

Talazoparib is sold by Pfizer Labs under the brand name Talzenna™. Talzenna™ is sold in capsule form at 1 mg. The dosage is 1 mg taken orally. Dosing continues until disease progression or unacceptable toxicity. This dosing regimen is referred to herein as the “normal” human dosing regimen for Talzenna™, regardless of the disorder treated. Any dosing regimen having a shorter duration (e.g., 21 days) or involving the administration of less Talzenna™ (e.g., 0.5 mg/day) is referred to herein as a “reduced” human dosing regimen. Examples of reduced human dosing regimens include the following: (i) 0.9 mg/day; (ii) 0.8 mg/day; (iii) 0.7 mg/day; (iv) 0.6 mg/day; (v) 0.5 mg/day; (vi) 0.4 mg/day; (vii) 0.3 mg/day; (viii) 0.2 mg/day; or (ix) 0.1 mg/day.

The terms “normal” human dosing regimen and “reduced” human dosing regimen also apply, mutatis mutandis, to any other PARP inhibitor with respect to its approved or otherwise customary dosing regimen.

### Example 9—²²⁵Ac-HuM195—Normal and Reduced Dosing Regimens

For an agent such as an antibody labeled with an alpha-emitting isotope, the majority of the drug administered to a subject typically consists of non-labeled antibody, with the minority being the labeled antibody. Doses of labeled agent used in connection with this invention include, for example, a single administration, and two or more administrations (i.e., fractions). The amount administered in each dose can be measured, for example, by labeled radiation activity (e.g., μCi/kg) or antibody weight (e.g., μg/kg or μg/m²).

In the case of ²²⁵Ac-HuM195, the “normal” human dosing regimen (regardless of the disorder treated), as this term is used herein, includes either of the following: (i) 4.0 μCi/kg administered fractionally in multiple administrations over no less than 1 week apart between doses; or (ii) 4.0 μCi/kg when delivered in a single administration.

A dosing regimen involving the administration of less ²²⁵Ac-HuM195 (e.g., 2.0 μCi/kg when delivered in a single administration) is referred to herein as a “reduced” human dosing regimen. Additional reduced human dosing regimens include, for example: (i) 2×<0.25 μCi/kg, 2×0.25 μCi/kg, 2×<0.5 μCi/kg, 2×0.5 μCi/kg, 2×<0.75 μCi/kg, 2×0.75 μCi/kg, 2×<1.0 μCi/kg, 2×1.0 μCi/kg, 2×<1.25 μCi/kg, 2×1.25 μCi/kg, 2×<1.5 μCi/kg, or 2×1.5 μCi/kg, where the fractions are administered one week apart; or (ii) 0.25 μCi/kg, 0.5 μCi/kg, 0.75 μCi/kg, 1.0 μCi/kg, 1.25 μCi/kg, 1.5 μCi/kg, 1.75 μCi/kg, 2.0 μCi/kg, 2.5 μCi/kg, 3.0 μCi/kg or 3.5 μCi/kg when delivered in a single administration. As a further example, reduced human dosing regimens of ²²⁵Ac-HuM195 include those corresponding to 25%, 50% or 75% of the normal dosing regimen.

The terms “normal” human dosing regimen and “reduced” human dosing regimen also apply, mutatis mutandis, to any other alpha-emitting isotope-labeled agent with respect to its approved or otherwise customary dosing regimen.

### Example 10—Dosing Scenario I for ²²⁵Ac-HuM195 and One of Olaparib, Niraparib, Rucaparib or Talazoparib

A human AML patient is treated according to the following regimen. One of olaparib, niraparib, rucaparib or talazoparib (referred to in this Example as “PARPi”) is orally administered according to its normal dosing regimen, accompanied by intravenous administration of ²²⁵Ac-HuM195 according to its normal dosing regimen (either single or fractional administration). In this Example and the others where applicable, the dosing regimens include the following embodiments, by way of example: (a) the PARPi and antibody radioconjugate are administered concurrently, wherein (i) each is administered beginning on the same day, (ii) the antibody is administered in a single dose or fractionated doses not less than one week apart, and (iii) the PARPi is administered daily or twice daily (as appropriate), and for a duration equal to or exceeding that of the antibody administration; or (b) the PARPi and antibody radioconjugate are administered concurrently, wherein (i) the PARPi administration precedes antibody radioconjugate administration by at least one week, (ii) the antibody is administered in a single dose or fractionated doses not less than one week apart, and (iii) the PARPi is administered daily or twice daily (as appropriate), and for a duration equal to or exceeding that of the antibody administration.

Also envisioned is the treatment of an experimental mouse model according to the treatment regimen in this scenario, whereby the appropriate dosing regimens are commensurate with mouse body weight and tumor xenograft size.

### Example 11—Dosing Scenario II for ²²⁵Ac-HuM195 and One of Olaparib, Niraparib, Rucaparib or Talazoparib

A human AML patient is treated according to the following regimen. One of olaparib, niraparib, rucaparib or talazoparib (referred to in this Example as “PARPi”) is orally administered according to its normal dosing regimen, accompanied by intravenous administration of ²²⁵Ac-HuM195 according to a reduced dosing regimen (either single or fractional administration). In one embodiment, the reduced dosing regimen of ²²⁵Ac-HuM195 is (i) 2×0.5 μCi/kg, 2×1.0 μCi/kg, or 2×1.5 μCi/kg, where the fractions are administered one week apart; or (ii) 1×0.5 μCi/kg, 1×1.0 μCi/kg, 1×2.0 μCi/kg, or 1×3.0 μCi/kg, for a single administration. In another embodiment, the reduced human dosing regimen of olaparib, niraparib, rucaparib or talazoparib includes that corresponding to 25%, 50% or 75% of its respective normal dosing regimen.

Also envisioned is the treatment of an experimental mouse model according to the treatment regimen in this scenario, whereby the appropriate dosing regimens are commensurate with mouse body weight and tumor xenograft size.

### Example 12—Dosing Scenario III for ²²⁵Ac-HuM195 and One of Olaparib, Niraparib, Rucaparib or Talazoparib

A human AML patient is treated according to the following regimen. One of olaparib, niraparib, rucaparib or talazoparib (referred to in this Example as “PARPi”) is orally administered according to a reduced dosing regimen, accompanied by intravenous administration of the normal dosing regimen of ²²⁵Ac-HuM195 (either single or fractional administration). In one embodiment, the reduced dosing regimen of PARPi is one of the following: (i) where the PARPi is olaparib, the reduced dosing regimen is 550 mg/day, 500 mg/day, 450 mg/day, 400 mg/day, 350 mg/day, 300 mg/day, 250 mg/day, 200 mg/day, 150 mg/day, 100 mg/day or 50 mg/day; (ii) where the PARPi is niraparib, the reduced dosing regimen is 250 mg/day, 200 mg/day, 150 mg/day, 100 mg/day or 50 mg/day; (iii) where the PARPi is rucaparib, the reduced dosing regimen is 1,150 mg/day, 1,100 mg/day, 1,050 mg/day, 1,000 mg/day, 950 mg/day, 900 mg/day, 850 mg/day, 800 mg/day, 750 mg/day, 700 mg/day, 650 mg/day, 600 mg/day, 550 mg/day, 500 mg/day, 450 mg/day, 400 mg/day, 350 mg/day, 300 mg/day, 250 mg/day, 200 mg/day, 150 mg/day or 100 mg/day; and (iv) where the PARPi is talazoparib, the reduced dosing regimen is 0.9 mg/day, 0.8 mg/day, 0.7 mg/day, 0.6 mg/day, 0.5 mg/day, 0.4 mg/day, 0.3 mg/day, 0.2 mg/day or 0.1 mg/day. In another embodiment, the reduced human dosing regimen of olaparib, niraparib, rucaparib or talazoparib includes that corresponding to 25%, 50% or 75% of its respective normal dosing regimen.

Also envisioned is the treatment of an experimental mouse model according to the treatment regimen in this scenario, whereby the appropriate dosing regimens are commensurate with mouse body weight and tumor xenograft size.

### Example 13—Dosing Scenario IV for ²²⁵Ac-HuM195 and One of Olaparib, Niraparib, Rucaparib or Talazoparib

A human AML patient is treated according to the following regimen. One of olaparib, niraparib, rucaparib or talazoparib (referred to in this Example as “PARPi”) is orally administered according to a reduced dosing regimen, accompanied by intravenous administration of a reduced dosing regimen of ²²⁵Ac-HuM195 (either single or fractional administration). In one embodiment, (a) the reduced dosing regimen of ²²⁵Ac-HuM195 is one of (i) 2×0.5 μCi/kg, 2×1.0 μCi/kg, or 2×1.5 μCi/kg, where the fractions are administered one week apart; or (ii) 1×0.5 μCi/kg, 1×1.0 μCi/kg, 1×2.0 μCi/kg, or 1×3.0 μCi/kg, for a single administration, and (b) the reduced dosing regimen of PARPi is one of the following: (i) where the PARPi is olaparib, the reduced dosing regimen is 550 mg/day, 500 mg/day, 450 mg/day, 400 mg/day, 350 mg/day, 300 mg/day, 250 mg/day, 200 mg/day, 150 mg/day, 100 mg/day or 50 mg/day; (ii) where the PARPi is niraparib, the reduced dosing regimen is 250 mg/day, 200 mg/day, 150 mg/day, 100 mg/day or 50 mg/day; (iii) where the PARPi is rucaparib, the reduced dosing regimen is 1,150 mg/day, 1,100 mg/day, 1,050 mg/day, 1,000 mg/day, 950 mg/day, 900 mg/day, 850 mg/day, 800 mg/day, 750 mg/day, 700 mg/day, 650 mg/day, 600 mg/day, 550 mg/day, 500 mg/day, 450 mg/day, 400 mg/day, 350 mg/day, 300 mg/day, 250 mg/day, 200 mg/day, 150 mg/day or 100 mg/day; and (iv) where the PARPi is talazoparib, the reduced dosing regimen is 0.9 mg/day, 0.8 mg/day, 0.7 mg/day, 0.6 mg/day, 0.5 mg/day, 0.4 mg/day, 0.3 mg/day, 0.2 mg/day or 0.1 mg/day. In another embodiment, the reduced human dosing regimen of olaparib, niraparib, rucaparib or talazoparib includes that corresponding to 25%, 50% or 75% of its respective normal dosing regimen.

Also envisioned is the treatment of an experimental mouse model according to the treatment regimen in this scenario, whereby the appropriate dosing regimens are commensurate with mouse body weight and tumor xenograft size.

### Example 14—²²⁵Ac-Trastuzumab

In one embodiment, the method for preparing ²²⁵Ac-trastuzumab is that for preparing ²²⁵Ac-HuM195 as described in Examples 3 and 4 above, wherein trastuzumab is substituted for HuM195. Further, each of the ²²⁵Ac-HuM195 dosing regimens (alone and in conjunction with PARPi) set forth in this application is also envisioned, mutatis mutandis, and without limitation, for ²²⁵Ac-trastuzumab.

### REFERENCES

1. Woodhouse, et al., 2008. DNA Rep. (Amst.) 7 (6), 932-940.
2. Krishnakumar, and Kraus, 2010. Mol. Cell 39 (1), 8-24.
3. Murai, et al., 2012. Cancer Res. 72 (21), 5588-5599.
4. Murai, J., et al., 2014. Mol Cancer Ther. 13 (2), 433-443.
5. Lord and Ashworth. 2017. Science 355, 1152-1158.
6. Drean, et al., 2016. Critical Rev in Oncol/hema. 108 (2016) 73-85.
7. Sizemore, et al., 2018. Mol Cancer Res., 16(7):1092-1102.
8. Brandwein, et al., 2007. Leukemia. 21:821-4.
9. Kiang, et al., Adaptive Medicine 2(1):1-10, 2010.
10. Co, et al., J. Immunol. 148:1149-1154, 1992.
11. Scheinberg, et al., U.S. Pat. No. 6,683,162.
12. Gansow, et al., U.S. Pat. No. 4,923,985.
13. McDevitt, Applied Radiation and Isotope, 57 (2002), 841-847.

1. A method for treating a subject afflicted with cancer, comprising administering to the subject (i) a poly-ADP ribose polymerase (“PARP”) inhibitor in conjunction with (ii) a radioisotope-labeled agent that targets cancer cells in the subject, wherein the amounts of the PARP inhibitor and labeled agent, when administered in conjunction with one another, are therapeutically effective.

2. The method of claim 1, wherein the subject is human.

3. The method of claim 1, wherein the cancer is a solid tumor.

4. The method of claim 1, wherein the cancer is selected from the group consisting of breast cancer, ovarian cancer, prostate cancer, lung cancer, squamous cell carcinoma of the head and neck, gastric cancer, pancreatic cancer, brain cancer, liver cancer, sarcoma and melanoma.

5. The method of claim 1, wherein the cancer is selected from the group consisting of breast cancer and ovarian cancer.

6. The method of any of claim 1, wherein the subject possesses a deleterious BRCA1/2 mutation.

7. The method of any of claim 1, wherein the subject does not possess a deleterious BRCA1/2 mutation.

8. The method of any of claim 1, wherein the PARP inhibitor is selected from the group consisting of olaparib, niraparib, rucaparib and talazoparib.

9. (canceled)

10. (canceled)

11. The method of claim 1, wherein the cancer is a hematologic malignancy.

12. The method of claim 1, wherein the hematologic malignancy is selected from the group consisting of acute myeloid leukemia, myelodysplastic syndrome and multiple myeloma.

13. (canceled)

14. The method of claim 1, wherein the radioisotope-labeled agent is an anti-CD33 antibody labeled with an alpha-emitting isotope.

15. The method of claim 1, wherein
the radioisotope-labeled agent is ²²⁵Ac-labeled HuM195.

16. (canceled)

17. A method for treating a human subject afflicted with acute myeloid leukemia, comprising administering to the subject (i) a PARP inhibitor selected from the group consisting of olaparib, niraparib, rucaparib and talazoparib in conjunction with (ii) ²²⁵Ac-labeled HuM195, wherein the amounts of the PARP inhibitor and ²²⁵Ac-labeled HuM195, when administered in conjunction with one another, are therapeutically effective.

18. A method for inducing the death of a cancer cell, comprising contacting the cell with (i) a PARP inhibitor in conjunction with (ii) a radioisotope-labeled agent that targets the cancer cell, wherein the amounts of PARP inhibitor and radiolabeled agent, when contacted with the cell in conjunction with one another, are effective to induce the cell's death.

19. The method of claim 18, wherein the cancer cell is a human cancer cell.

20. The method of claim 18, wherein the cancer cell is selected from the group consisting of a breast cancer cell and an ovarian cancer cell.

21. The method of claim 18, wherein the cancer cell does not possess a deleterious BRCA1/2 mutation.

22. The method of claim 18, wherein the PARP inhibitor is selected from the group consisting of olaparib, niraparib, rucaparib and talazoparib.

23. (canceled)

24. (canceled)

25. (canceled)

26. The method of claim 18, wherein the cancer cell is a hematologic cancer cell.

27. The method of claim 18, wherein the hematologic cancer cell is selected from the group consisting of an acute myeloid leukemic cell, a myelodysplastic syndrome cell, and a multiple myeloma cell.

28. The method of claim 18, wherein the PARP inhibitor is selected from the group consisting of olaparib, niraparib, rucaparib and talazoparib.

29. The method of any of claim 18, wherein the radioisotope-labeled agent is an anti-CD33 antibody labeled with an alpha-emitting isotope.

30. The method of any of claim 18, wherein the radioisotope-labeled agent is ²²⁵Ac-labeled HuM195.

31. A method for inducing the death of an acute myeloid leukemic cell, comprising contacting the cell with (i) a PARP inhibitor selected from the group consisting of olaparib, niraparib, rucaparib and talazoparib in conjunction with (ii) ²²⁵Ac-labeled HuM195, wherein the amounts of PARP inhibitor and ²²⁵Ac-labeled HuM195, when contacted with the cell in conjunction with one another, are effective to induce the cell's death.

32. (canceled)

33. (canceled)

34. (canceled)


SEQUENCE LISTING (4 sequences)

SEQ ID NO: 1 - DNA, 1952 bp, Artificial Sequence
  Description of Artificial Sequence Synthetic polynucleotide
tctagaccaccatggagaaagacacactcctgctatgggtcctacttctctgggttccaggttccacaggtgacattcagatgacccagtctccgagctctctgtccgcatcagtaggagacagggtcaccatcacatgcagagccagcgaaagtgtcgacaattatggcattagctttatgaactggttccaacagaaacccgggaaggctcctaagcttctgatttacgctgcatccaaccaaggctccggggtaccctctcgcttctcaggcagtggatctgggacagacttcactctcaccatttcatctctgcagcctgatgacttcgcaacctattactgtcagcaaagtaaggaggttccgtggacgttcggtcaagggaccaaggtggagatcaaacgtaagtagaatccaaagtctagaaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgctctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcacctgtggtttctctctttcctcatttaataattattatctgttgttttaccaactactcaatttctcttataagggactaaatatgtagtcatcctaaggcgcataaccatttataaaaatcatccttcattctattttaccctatcatcctctgcaagacagtcctccctcaaacccacaagccttctgtcctcacagtcccctgggccatggtaggagagacttgcttccttgttttcccctcctcagcaagccctcatagtcctttttaagggtgacaggtcttacagtcatatatcctttgattcaattccctgagaatcaaccaaagcaaatttttcaaaagaagaaacctgctataaagagaatcattcattgcaacatgatataaaataacaacacaataaaagcaattaaataaacaaacaatagggaaatgtttaagttcatcatggtacttagacttaatggaatgtcatgccttatttacatttttaaacaggtactgagggactcctgtctgccaagggccgtattgagtactttccacaacctaatttaatccacactatactgtgagattaaaaacattcattaaaatgttgcaaaggttctataaagctgagagacaaatatattctataactcagcaatcccacttctaggatcc

SEQ ID NO: 2 - protein, 238 aa, Artificial Sequence
  Description of Artificial Sequence Synthetic polypeptide
MEKDTLLLWVLLLWVPGSTGDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 3246 bp, Artificial Sequence
  Description of Artificial Sequence Synthetic polynucleotide
tctagaccaccatgggatggagctggatctttctcttcctcctgtcaggaactgctggcgtccactctcaggttcagctggtgcagtctggagctgaggtgaagaagcctgggagctcagtgaaggtttcctgcaaagcttctggctacaccttcactgactacaacatgcactgggtgaggcaggctcctggccaaggcctggaatggattggatatatttatccttacaatggtggtaccggctacaaccagaagttcaagagcaaggccacaattacagcagacgagagtactaacacagcctacatggaactctccagcctgaggtctgaggacactgcagtctattactgcgcaagagggcgccccgctatggactactggggccaagggactctggtcactgtctcttcaggtaagaatggcctctagaccaccatgggatggagctttctggggcaggccaggcctgaccttggctttggggcagggagggggctaaggtgaggcaggtggcgccagccaggtgcacacccaatgcccatgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagcccccgctccccgggctctcgcggtcgcacgaggatgcttggcacgtaccccctgtacatacttcccgggcgcccagcatggaaataaagcacccagcgctgccctgggcccctgcgagactgtgatggttctttccacgggtcaggccgagtctgaggcctgagtggcatgagggaggcagagcgggtcccactgtccccacactggcccaggctgtgcaggtgtgcctgggccgcctagggtggggctcagccaggggctgccctcggcagggtgggggatttgccagcgtggccctccctccagcagcacctgccctgggctgggccacgggaagccctaggagcccctggggacagacacacagcccctgcctctgtaggagactgtcctgttctgtgagcgccctgtcctccgacctccatgcccactcgggggcatgcctagtccatgtgcgtagggacaggccctccctcacccatctacccccacggcactaacccctggctgccctgcccagcctcgcacccgcatggggacacaaccgactccggggacatgcactctcgggccctgtggagggactggtgcagatgcccacacacacactcagcccagacccgttcaacaaaccccgcactgaggttggccggccacacggccaccacacacacacgtgcacgcctcacacacggagcctcacccgggcgaactgcacagcacccagaccagagcaaggtcctcgcacacgtgaacactcctcggacacaggcccccacgagccccacgcggcacctcaaggcccacgagcctctcggcagcttctccacatgctgacctgctcagacaaacccagccctcctctcacaagggtgcccctgcagccgccacacacacacaggggatcacacaccacgtcacgtccctggccctggcccacttcccagtgccgcccttccctgcaggatcc

SEQ ID NO: 4 - protein, 465 aa, Artificial Sequence
  Description of Artificial Sequence Synthetic polypeptide
MGWSWIFLFLLSGTAGVHSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK